(19) United States
(12) Patent Application Publication
(10) Pub. No.: US 2009/0192436 A1
(43) Pub. Date: Jul. 30, 2009

(54) APPLICATOR HAVING PLUNGER WITH GRIPPING ELEMENTS

(76) Inventors: Nancy Karapasha, Cincinnati, OH (US); Dean Jeffrey Daniels, Cincinnati, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/019,301

(22) Filed: Jan. 24, 2008

(51) Int. Cl. A61F 13/20

(52) U.S. Cl. 604/15; 604/11; 604/385.18; 604/385.17

(57) ABSTRACT

The present invention provides applicators for inserting feminine hygiene articles, such as and pessaries into a user's body.

### FIELD OF THE INVENTION

The present invention relates generally to applicators, more specifically to applicators having plungers with gripping elements.

### BACKGROUND OF THE INVENTION

An applicator for feminine hygiene articles, such as tampons and pessaries, typically comprises two components, namely a barrel and a plunger that is configured to telescopically slide within the barrel. The feminine hygiene article to be inserted in the vaginal canal is positioned in the barrel of the applicator. The barrel has a first end for insertion of the feminine hygiene article, and a second end for receipt of the plunger. To use the applicator, the user will position the insertion end of the barrel at or near the vaginal opening, grasp the barrel, and move or slide the plunger in the barrel towards the insertion end of the barrel to insert the feminine hygiene article.

Feminine hygiene articles, specifically tampons due to their design, exert a pressure or friction force on the inside wall of the barrel. To overcome the resistance to insertion of a feminine hygiene article finger grips have been added to a region of the barrel. This allows a user to have a secure grip on the barrel to ease insertion. However, focusing all the users force on one area of the barrel can cause deformation of the barrel. Such deformation can further inhibit insertion of the feminine hygiene article causing the feminine hygiene article to be improperly inserted into the vaginal canal, or not inserted at all. Further, not all users insert a feminine hygiene article in the same manner; as such some users may not even use the finger grip region.

In certain applicators, the plunger has been modified from a symmetrical tube to assist in insertion. For, example the walls of the plunger have been thickened or formed from relatively stiff material to allow it to provide increased pressure for insertion. In other applicators, the plunger has been widened at one or both of its end regions. Widening the plunger at the withdrawal end is thought to provide an increased surface area for the user to place their thumb to move the plunger for insertion, or to limit insertion distance. Widening the insertion end of the plunger is believed to prevent the plunger from separating from the barrel. However, the plunger has not been modified to increase the ease with which a user can grip it. With the exception of the feminine hygiene article the plunger is the only part of the applicator which moves. The plunger determines the force at which the feminine hygiene article is inserted, and as such contributes to the placement of the feminine hygiene article within the vaginal canal. Therefore, control of the movement of the plunger can provide greater control of the placement of the feminine hygiene article within the vaginal canal.

Consequently, it would be desirable to provide an applicator with a plunger that grants a user increased control over insertion of a feminine hygiene article.

### SUMMARY OF THE INVENTION

A feminine hygiene applicator is provided which comprises a barrel having an outer surface, and a plunger having an outer surface. The plunger comprises at least one gripping element.

A feminine hygiene applicator is provided which comprises a barrel, a moveable grip region, and a plunger. The barrel is rotatably connected to the moveable grip region. Further, the barrel is adapted to rotate relative to the moveable grip region, and the plunger comprises at least one gripping element.

### BRIEF DESCRIPTION OF THE DRAWINGS

FIG. 1 is a perspective view of one embodiment of the present invention.

FIG. 2 is a perspective view of one embodiment of the present invention.

FIG. 3 is a perspective view of one embodiment of the present invention.

FIG. 4 is a perspective view of one embodiment of the present invention.

FIG. 5 is a perspective view of one embodiment of the present invention.

FIG. 6 is a perspective view of one embodiment of the present invention.

FIG. 7 is a cross-sectional view along section 7-7 of FIG. 6.

FIG. 8 is a perspective view of one embodiment of the present invention.

FIG. 9 is a perspective view of one embodiment of the present invention.

FIG. 10 is a cross-sectional view along section 10-10 of FIG. 9.

FIG. 11 is a cross-sectional view of one embodiment of the present invention.

FIG. 12 is a cross-sectional view of one embodiment of the present invention.

FIG. 13 is a cross-sectional view of one embodiment of the present invention.

FIG. 14 is a cross-sectional view of one embodiment of the present invention.

FIG. 15 is a partial cut-away view of one embodiment of the present invention.

FIG. 16 is a perspective view of one embodiment of the present invention.

FIG. 17 is a perspective view of one embodiment of the present invention.

FIG. 18 is a perspective view of one embodiment of the present invention.

### DETAILED DESCRIPTION OF THE INVENTION

The present invention relates to one or more applicators for inserting feminine hygiene articles, such as tampons and pessaries into a user's body. Such an applicator must overcome the frictional resistance to insertion created by the feminine hygiene article passing through not only the inner surface of the applicator's barrel, but also the vaginal opening and vaginal canal. A feminine hygiene article, such as a tampon may expand within the barrel of an applicator due to age or acquisition of ambient moisture, and exert pressure on the inside surface of the barrel. Additionally, the absorbent surface of a feminine hygiene article will often partially adhere to the surface of the labia and the vaginal walls. Further, insertion of a feminine hygiene article is often done out of the users direct view. These factors need to be overcome to ensure proper placement of a feminine hygiene article. The present invention provides greater control over the insertion of feminine hygiene articles by providing an applicator having a plunger comprising gripping elements. The gripping elements on or in the plunger grant a user an improved grip on the plunger allowing greater control over insertion of a feminine hygiene article. A plunger having gripping elements also provides greater force generation, as the user has an improved grip on the plunger, allowing the applicator to overcome the friction generated by the insertion of the feminine hygiene article. In addition, the gripping elements reduce the surface area of the plunger in contact the inner surface of the barrel, thereby decreasing friction between the outer surface of the plunger and the inner surface of the barrel, and easing insertion of a feminine hygiene article.

As used herein, “feminine hygiene article” refers to articles that absorb, block, or contain body exudates discharged from the body, and which may be placed within or in proximity to the body of a user. Examples of feminine hygiene articles include tampons and pessaries.

As used herein, the term “tampon” refers to any type of absorbent structure that can be inserted into the vaginal canal or other body cavity, for purposes such as the absorption of fluid, to aid in wound healing, or for the delivery of materials, such as moisture or active materials, for instance medicaments. In general, the term “tampon” is used to refer to a finished tampon after the compression or shaping process. However, dry expanding tampons may also be used.

As used herein, the term “pessary” refers to any type of substantially non-absorbent structure used for the purpose of reducing urine leakage, such as by supporting a prolapsed uterus or bladder. Such pessaries can have any variety of shapes and sizes including cylinder, ovate, spherical, tubular, annual rings, “U” shaped, cup shaped, rings, cubes or donut shaped, and can function in any suitable manner, such as by direct application of support, lever force, expansion of the device by selection of material or by inflation of the device.

As used herein, the term “vaginal canal” refers to the internal genitalia of the human female in the pudendal region of the body. The terms “vaginal canal” or “within the vagina” as used herein are intended to refer to the space located between the introitus of the vagina (sometimes referred to as the sphincter of the vagina) and the cervix.

As used herein, “applicator” refers to a device or implement that facilitates the insertion of a tampon or pessary into an external orifice of a user. Exemplary applicators include tube and plunger and compact applicators.

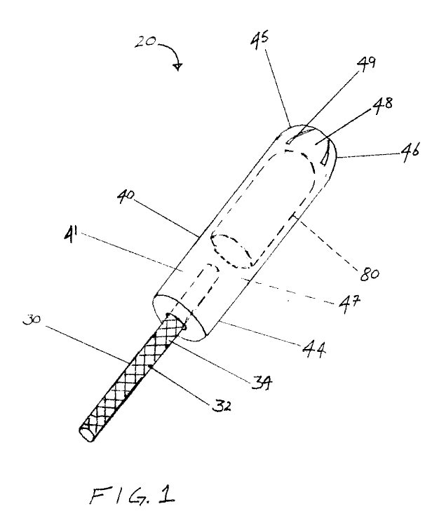

FIG. 1 shows a plan view of one embodiment of the invention. The applicator 20 has a plunger 30 and a barrel 40. The barrel 40 has an outer surface 41, an inner surface 47, a forward, insertion end 45, and an opposite plunger receiving end 44. The insertion end 45 of the barrel 40 can be open or can have a dome shape. In certain embodiments, as shown in FIG. 1, the insertion end 45 may have a hemispherical, dome-shaped tip 46. The tip 46 may include one or more petals 48 that, in certain embodiments, may be formed by one or more slits 49. The petals 48 may be flexible, allowing a feminine hygiene article, in this instance a tampon 80, to be ejected through the petals 48 when the plunger 30 is pressed against the withdrawal end 82 of the tampon 80 within the barrel 40. The plunger 30 can be telescopically or slidably mounted in the barrel 40. The plunger 30 is configured to eject a feminine hygiene article. One or more gripping elements 32 are disposed on or in the outer surface 34 of the plunger 30.

In certain embodiments, to reduce friction or increase strength, the outer surface 34 of the plunger 30 or the inner surface 47 of the barrel 40 may be coated with a coating material. Suitable coating materials include, for example, cellophane, cellulose, epoxy, lacquer, nitrocellulose, nylon, polyester, polylactide, polyolefin, polyurethane, polyvinyl alcohol, polyvinyl chloride, silicone, wax, mica, or any combinations thereof.

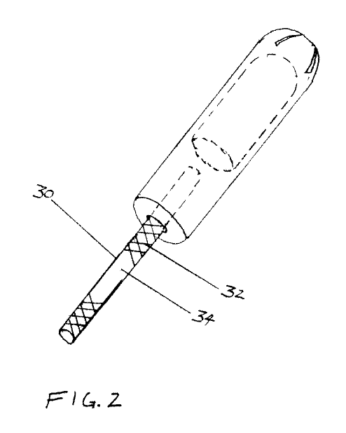

A gripping element may be in any suitable form for example, an embossment, tread, rib, projection, slit, groove, perforation, depression, lance, abrasive material, pressure sensitive adhesive, high wet coefficient friction material, or any combination thereof. A gripping element may have any suitable shape, such as circular, oval, square, rectangular, triangular, polygonal, linear, curved, irregular, character, letter, symbol, number, or combinations thereof. A gripping element may be raised above the outer surface, depressed below or through the outer surface, angled away or towards the outer surface, or any combinations thereof. Gripping elements may be patterned or arranged in any configuration, and in any number suitable for lowering friction or creating an enhanced gripping area for a user's fingers. For example, as shown in FIG. 1 and FIG. 2 the gripping elements 32 may be arranged in a grid pattern, and may be disposed on or in substantially the entire outer surface 34 of the plunger 30 (FIG. 1) or only partially on or in the outer surface 34 of the plunger 30 (FIG. 2). Further, gripping elements 32 may be any suitable color.

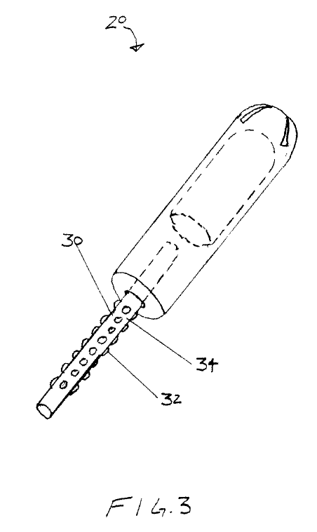

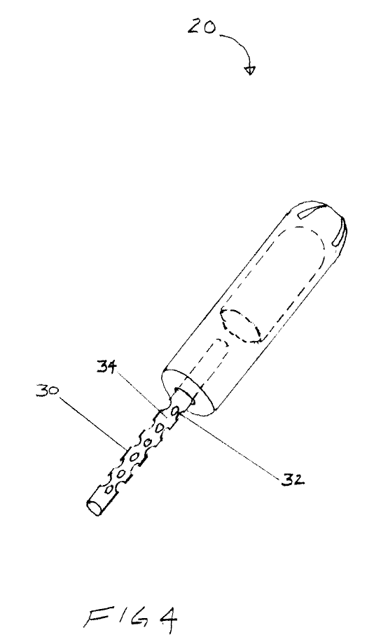

In certain embodiments, as shown in FIG. 3 and FIG. 4 the plunger 30 of an applicator 20 may have one or more gripping elements 32 disposed on or in the outer surface 34 of the plunger 30, wherein the gripping elements 32 have a circular or domed shape. The circular shaped gripping elements 32 may be raised above the outer surface 34 (FIG. 3), depressed below the outer surface 34 (FIG. 4), through or substantially through the outer surface 34, or combinations thereof.

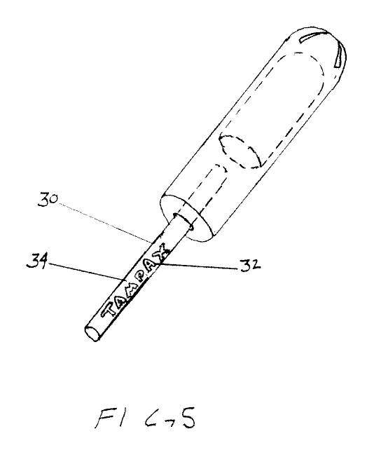

As shown in FIG. 5 the gripping elements 32 may be in the shape of a logo or trademark, such as TAMPAX®, from the Procter and Gamble Company of Cincinnati, Ohio. The use of gripping elements 32 in the shape of a logo or trademark would provide brand identity to a consumer, even after the applicator has been removed from its package. The logo or trademark may be in any font or size suitable for disposition on or in the outer surface 34 of the plunger 30. The trademark or logo shaped gripping elements 32 may be raised above the outer surface 34, depressed below the outer surface 34, through or substantially through the outer surface 34, or combinations thereof.

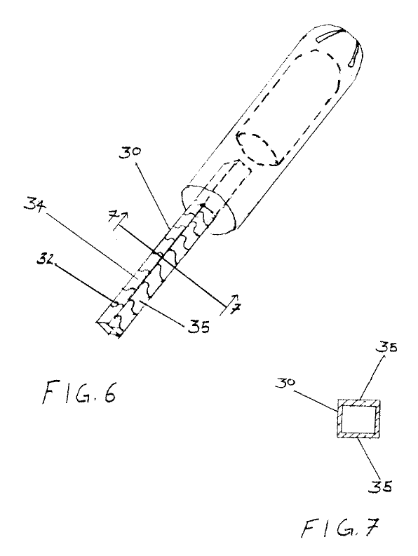

FIG. 6 shows that the plunger 30 may have cross-sectional shapes other than circular. As demonstrated in FIG. 6, the outer surface 34 of a plunger 30 may have one or more substantially flat planes 35, having gripping elements 32 disposed thereon or therein. FIG. 7, which is a cross-section of the plunger of FIG. 6, shows two opposing sets of substantially flat planes 35, forming a square or rectangular cross-section of the plunger. However, there could be other plunger embodiments having substantially flat planes with cross-sections other than square or rectangular, such as triangular, hexagonal, a flattened circular or ovoid cross-section, or any other combination of substantially flat planes suitable for use on a plunger. It is believed that a plunger having one or more substantially flat planes provides a greater surface area for a user to place their fingers on, as compared to a plunger with a circular or ovoid cross-section. The greater surface area in combination with gripping elements provides a user with enhanced control and force generation.

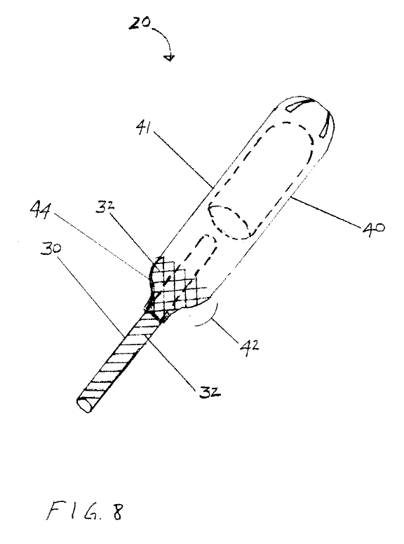

In certain embodiments, as shown in FIG. 8, in addition to a plunger 30 having gripping elements 32, an applicator 20 may comprise a finger grip region 42 to enhance the gripping of the barrel 40. In certain embodiments the finger grip region 42 may be disposed about the circumference of the outer surface 41 of the barrel 40. The finger grip region 42 may also be disposed adjacent to or in close proximity to the plunger receiving end 44 of the barrel 40. In certain embodiments, as shown in FIG. 8 the finger grip region 42 may have one or more gripping elements 32 disposed thereon or therein. The gripping elements 32 are the same as described above with reference to the gripping elements 32 disposed on or in the plunger 30. The gripping elements of the plunger 30 may be same or differ in number, form, shape or size when compared to the gripping elements of the finger grip region 42.

When a user's fingers contact the gripping elements 32 on the plunger 30 and the finger grip region 42 the gripping elements 32 of both the plunger 30 and finger grip region 42 provide enhanced control and force generation. The grip elements 32 disposed on or in the plunger 30 and finger grip region 42 additionally provide increased ease of withdrawal of the barrel 40 from the vaginal opening. Therefore, a user can firmly control the insertion of the barrel 40 of the applicator 20 into the vaginal opening, the expulsion of the feminine hygiene article contained therein, and subsequent removal of the barrel 40, because of the gripping elements 32 disposed on or in the plunger 30 and finger grip region 42.

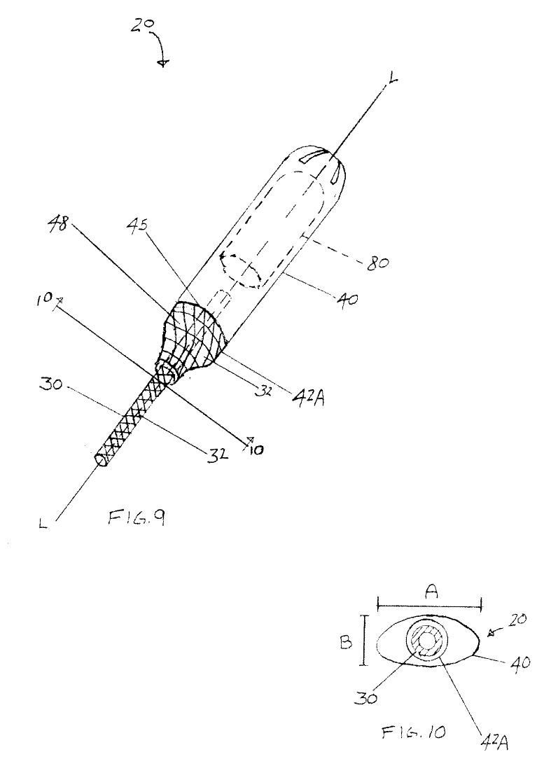

FIG. 9 shows a perspective view of one embodiment of the present invention. The applicator 20 comprises a plunger 30, a movable grip region 42A, a barrel 40, and a longitudinal axis “L”. The barrel 40 can be of any suitable shape or size for storing and ejecting a feminine hygiene article, which in this embodiment is a tampon 80. In this embodiment, the movable grip region 42A is rotatably connected to the barrel 40. For example, the barrel 40 can rotate relative to the movable grip region 42A, such as when the movable grip region 42A is held stationary by a user. As shown in FIG. 9, in certain embodiments, the barrel 40 and movable grip region 42A can be non-contiguous, for example, the barrel 40 and the movable grip region 42A can be separated by a gap 45, which can allow the barrel 40 to rotate relative to the movable grip region 42A. In certain embodiments, gripping elements 32 as described previously with reference to the plunger 30 may be disposed on or in the outer surface 48 of the moveable grip region 42A. The plunger 30 can be telescopically or slidably mounted in the barrel 40, movable grip region 42A, or both. The gripping elements 32 of the plunger 30 may be same or differ in number, shape, form or size when compared to the gripping elements 32 of the moveable grip region 42A.

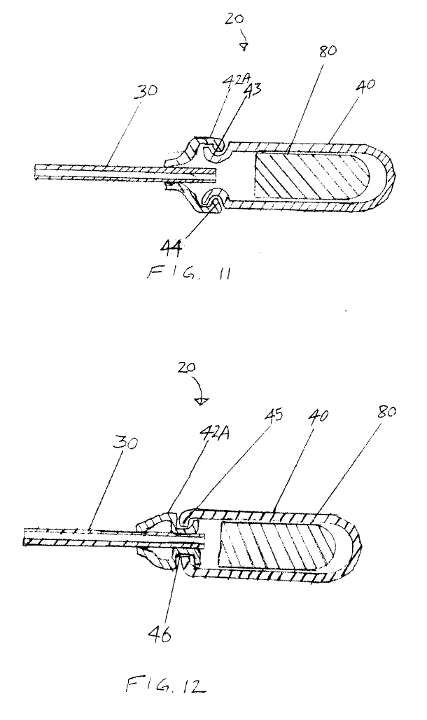

FIG. 10 shows a cross-sectional view along section 10-10 of FIG. 9. The applicator 20 shown has a plunger 30, a moveable grip region 42A, and a barrel 40. In this embodiment, the barrel 40 has an oval cross-section, while the plunger 30 and moveable grip region 42A have cylindrical cross-sections. The applicator 20 shown in FIG. 10 has a width A and a thickness B running perpendicular to the longitudinal axis L and to each other. In certain embodiments, the width A is larger than the thickness B.

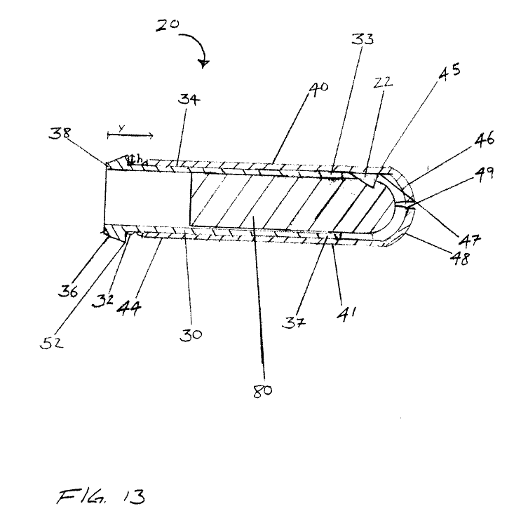

FIG. 11 and FIG. 12 show cross-sectional views along the longitudinal axis of an applicator as shown in FIG. 9. In each embodiment shown, the applicator 20 has a plunger 30, a moveable grip region 42A, and a barrel 40. As shown in FIG. 11 and FIG. 12, the moveable grip region 42A can be rotatably connected to the barrel 40. The moveable grip region 42A can be rotatably connected to the barrel 40 in any suitable manner. For example, the moveable grip region 42A can be rotatably connected to the barrel 40 such that the moveable grip region 42A is attached in the axial direction relative to the direction of tampon 80 insertion, but is rotatable with respect to the barrel 40. Suitable connecting means include, for example, one or more protrusions, indentations, tabs, channels, flanges, rims, rings, edges, ribs, collars, or other suitable means for rotatably connecting the barrel 40 to the moveable grip region 42A. As shown in FIG. 11 and FIG. 12, in certain embodiments, the moveable grip region 42A can attach to the barrel 40, such as, by using a combination of protrusions and indentations that can join the barrel 40 to the moveable grip region 42A in a manner that allows rotation of the moveable grip region 42A about the barrel 40. In certain embodiments, as shown in FIG. 11, the moveable grip region 42A can attach to the barrel 40 by using one or more indentations 43 on the barrel 40 and one or more protrusions 44 on the moveable grip region 42A. In certain other embodiments, as shown in FIG. 12, the moveable grip region 42A can attach to the barrel 40 by using one or more protrusions 45 on the barrel 40 and one or more indentations 46 on the moveable grip region 42A.

As such, the applicator shown in FIGS. 9-12 can comprise a barrel, a moveable grip region, and a plunger, wherein the barrel can rotate relative to the moveable grip region, such as, wherein the barrel of the applicator can rotate under body pressure relative to the moveable grip region to properly orient a feminine hygiene article within a user's vagina. This is because length, oriented in a front to back manner, is the longest dimension of the labia and vaginal opening, but the longest dimension of the vaginal canal is its width, which is in a side to side direction relative to a user's body. In certain embodiments, the feminine hygiene article can include a tampon that provides increased leakage protection upon proper orientation in the vagina, such as, a tampon with non-uniform expansion, for example a tampon having improved widthwise expansion compared to a tampon that can expand generally uniformly in more than one dimension. The tampon can be inserted into a user's body using an applicator as described herein such that the applicator positions the tampon with the widthwise expansion aligned with the width of the vagina. For instance, the user can grasp the applicator by placing their fingers on the movable grip region, insert the barrel containing the tampon into their vagina, depress the plunger to expel the article from the barrel into their vagina, and remove the applicator. If the user inserts the barrel such that the portion of the tampon having the largest potential widthwise expansion is oriented in a front to back manner or at an angle, the barrel can rotate within the user's body so that the tampon is in the correct position, for example, with the largest width in a side to side position, such that the maximum expansion of the tampon is aligned with the maximum width of the vagina.

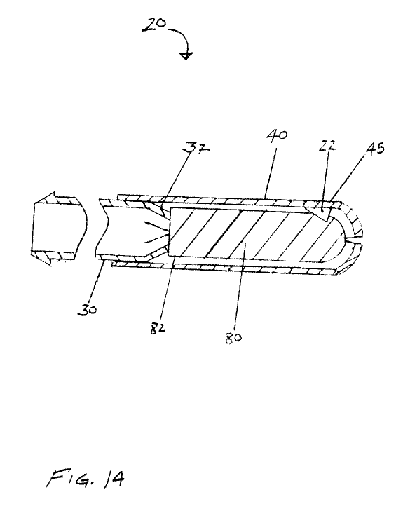

In certain embodiments, as shown in FIG. 13, the applicator 20 comprises a barrel 40. The barrel 40 has an outer surface 41, an inner surface 47, a forward, insertion end 45, and an opposite plunger receiving end 44. The insertion end 45 of the barrel 40 can be open or can have a dome shape. In certain embodiments, as shown in FIG. 13, the insertion end 45 may have a hemispherical, dome-shaped tip 46. The tip 46 may include one or more petals 48 that in certain embodiments may be formed by one or more slits 49. The petals 48 may be flexible, allowing a feminine hygiene article, in this instance a tampon 80, to be ejected through the petals 48 when the plunger 30 is pressed against the bottom of the tampon 80 within the barrel 40.

In certain embodiments, the barrel 40 may include one or more inward projections 22, which can be secured along the inner surface 47 of the barrel 40. The projections 22, as shown in FIG. 13, which may have a substantially flat right triangular configuration with the long side thereof extending at an acute angle from the inner surface 47 of the barrel 40 towards the insertion end 45. The projections 22 should extend far enough into the center of the barrel 40 to engage a feminine hygiene article, in this instance a tampon 80 disposed therein. Although one projection 22 is shown in FIG. 13 for engaging the tampon 80, this number may vary.

The applicator 20 illustrated in FIG. 13 further comprises a plunger 30, which serves to eject a tampon 80 positioned within the barrel 40. The plunger 30 has one or more gripping elements 32 disposed on or in its outer surface 34 as described previously. The plunger 30 is dimensioned to slidably move within the barrel 40. In certain embodiments, the plunger 30 is also slightly longer than the barrel 40 to assure complete ejection of the tampon 80. The insertion end 33 of the plunger 30 may also be provided with a means suitable for ejecting the tampon 80, in this instance foldable flaps 37, when the plunger 30 is retracted during operation, so as to eject the tampon 80 from the barrel 40, as explained in more detail below. Other examples of such suitable means include projections or corrugations.

The plunger 30 may have one or more longitudinal protrusions 36 disposed at or near the plunger's 30 withdrawal end 38. The longitudinal protrusions 36 protrude radially outward from the outer surface 34 of the plunger 30, and may extend completely or partially around the perimeter of the plunger 30. Referring to FIG. 13, the plunger 30 has the closest portion of a longitudinal protrusion 36 spaced a distance y from the withdrawal end 38 of the plunger 30 (in this instance the y distance is zero as the longitudinal protrusion 36 is positioned at the withdrawal end 38 of the plunger 30). In certain embodiments, the closest portion of the longitudinal protrusion 36 of the plunger 30 can be spaced from about 0 mm to about 7 mm from the withdrawal end 38 of the plunger 30. In certain embodiments, the closest portion of the longitudinal protrusion 36 of the plunger 30 can be spaced from about 2 mm to about 5 mm from the withdrawal end 38 of the plunger 30. The longitudinal protrusion 36 has a height dimension h as measured from the outer surface 34 of the plunger 30 to the highest point 52 of the longitudinal protrusion 36. The height dimension h of the longitudinal protrusion 36 may be about 0.25 mm to about 4 mm.

When the longitudinal protrusion 36 engages with the plunger receiving end 44 of the barrel 40, the longitudinal protrusion 36 acts as a stop and a signal to the user that the tampon 80 has been fully expelled. Further, the longitudinal protrusion 36 provides a visual cue to a user as to the proper placement of their fingers upon the plunger 30. In addition to providing a visual cue the longitudinal protrusion 36 provides a user with an improved grip, such that the plunger 30 can more easily be withdrawn from the barrel 40, and then more easily moved within the barrel 40 to expel the tampon 80.

The longitudinal protrusions 36 can be of any suitable number, shape, or size. Longitudinal protrusions 36 may take the form of a semi-hemispherical shape, multi-angular shape, bumps, pyramids, rings, and the like. Moreover, longitudinal protrusions 36 may form any pattern. For example, the longitudinal protrusions 36 may form the pattern of a row. The longitudinal protrusions 36 can be joined to the plunger 30 or formed in the plunger 30 in any way known in the art. The longitudinal protrusions 36 can also be integral with the plunger 30, for example, by molding the longitudinal protrusions 36 and the plunger 30 as a single piece. In other embodiments, the longitudinal protrusions 36 can be joined to the plunger 30 by friction fitting that snaps parts together, gluing, or melting. Longitudinal protrusions may also be formed by an embossing process.

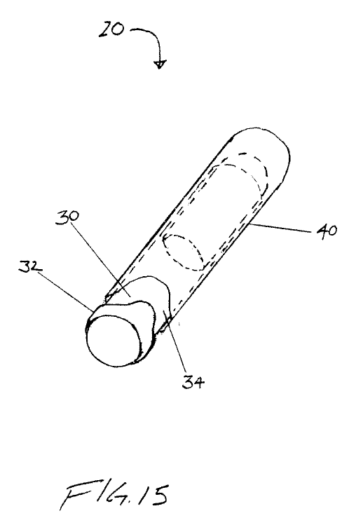

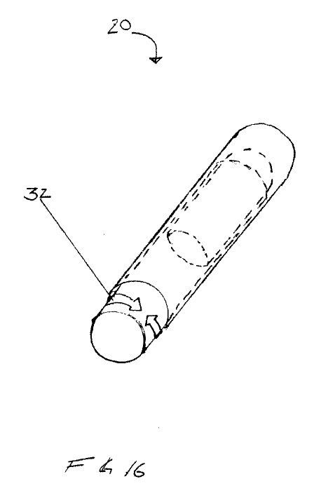

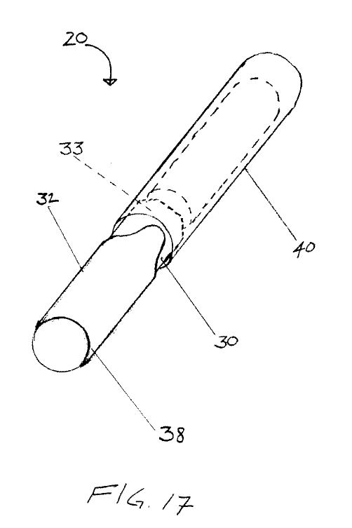

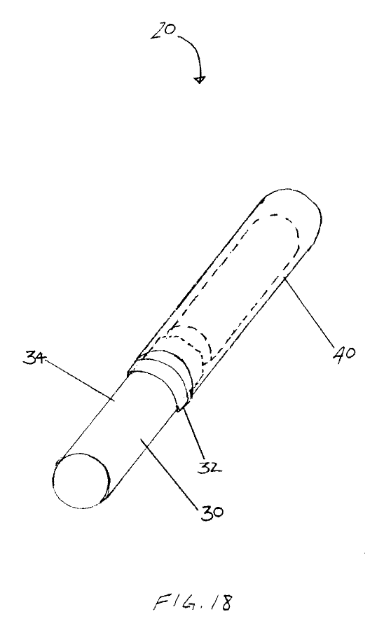

FIG, 13 shows the applicator 20 in its stored configuration, ready for packaging, with the plunger 30 being disposed almost entirely within the barrel 40. The tampon 80 is stored within the plunger 30, not directly in the barrel 40. In this configuration, the barrel 40 is telescoped over the plunger 30. As shown in FIG, 14, to operate the applicator 20, the barrel 40 is held while the plunger 30 is partially withdrawn therefrom (i.e. the plunger 30 is moved in the direction away from the insertion end 45 of the barrel 40). In certain embodiments, to help a user withdraw the plunger 30 from the barrel 40, gripping elements 32 may be disposed on or in the outer surface 34 of the plunger 30 to provide a visual signal as to where the user may place their fingers on the outer surface 34 of the plunger 30. For example, gripping elements 32 may have a different color than the surrounding outer surface 34 of a plunger 30, such as gripping elements 32 having a blue color against a white colored plunger 30 outer surface 34; gripping elements 32 may be disposed on or in the areas of the outer surface 34 of the plunger 30 where a user may place their fingers to withdraw the plunger 30 from the barrel 40 (FIG. 15); gripping elements 32 may have a shape that signals to a user where they may place their fingers, such as an arrow or characters (FIG. 16); or any combination thereof. Further, in certain embodiments, the gripping elements 32 may indicate to a user the distance a plunger 30 may be withdrawn from the barrel 40 to insert the tampon 80. For example, as shown in FIG. 17, the gripping elements 32 could, starting at the withdrawal end 38, extend in the direction of the insertion end 33 of the plunger 30 a distance that corresponds to the distance the plunger 30 may be withdrawn from the barrel 40 or, as shown in FIG. 18, the gripping elements 32 may be disposed at a location on or in the outer surface 34 of the plunger 30 that indicates the distance the plunger 30 may be withdrawn from the barrel 40. Referring back to FIG. 14, during the activating step, the tampon 80 remains in a fixed position relative to the barrel 40 by means of the projection 22, which acts to secure the tampon 80 in place, and restrain its movement in the rearward direction. The folded flaps 37 of the plunger 30 slide over the tampon 80. Once the folded flaps 37 are withdrawn past the withdrawal end 82 of the tampon 80, they close toward each other so as to be positioned behind the withdrawal end 82 of the tampon 80 for the start of the ejection operation.

The following description provides examples of components that may be used in the embodiments of the present invention described previously. The barrel can be constructed from any suitable material. Suitable materials include, for example, polyethylene, polypropylene, polybutylene, polystyrene, polyvinylchloride, polyacrylate, polymethacrylate, polyacrylnitril, polyacrylamide, polyamide, nylon, polyimide, polyester, polycarbonate, ethylene vinyl acetate, polyurethane, silicone, paper, paperboard, cardboard, cellulose, or combinations thereof. Examples of suitable materials are disclosed in U.S. Pat. Nos. 5,346,468 and 5,558,631. In certain embodiments, additives can be included in the material to alter or enhance certain material properties. Suitable additives include, for example, mold release agents, slip agents, surface energy modifiers, pearlescent agents, or any other suitable additives. In certain embodiments, the barrel can be coated with a substance to give it a high slip characteristic, for example wax, polyethylene, cellophane, clay, mica, combinations thereof, or any other lubricants that can facilitate comfortable insertion.

The barrel can be sized and configured to house a feminine hygiene article, such as, a tampon or pessary. In certain embodiments, the size of the barrel can be determined primarily by the dimensions of the feminine hygiene article. For example, the barrel can have inner diameters of about 5 mm to about 22 mm and a wall thickness of about 0.2 mm to about 2 mm. The inner diameter of the barrel can be greater than the diameter of the feminine hygiene article to prevent the barrel from interfering with the expulsion of the feminine hygiene article from the barrel. In certain embodiments, the inner diameter of the barrel can have varying diameters and shapes to conform to the profiled shape of the enclosed feminine hygiene article, such as a tampon. The barrel can have a length sufficient to house the feminine hygiene article prior to the expulsion of the feminine hygiene article from the applicator into the vagina.

The barrel may have any suitable cross-sectional shape, such as circular. In certain embodiments, the barrel can include a generally non-circular cross-sectional shape, such as an oval, rectangle, square, ellipse, oblong, or other suitable shapes. The barrel can have a cross-sectional shape that has a greater thickness than width or vice versa. In certain embodiments, the barrel can have a substantially uniform cross-section, such as having substantially the same cross-section along the length. In other embodiments, the barrel can have varying cross-sectional shapes, cross-sectional sizes, or both, such as a barrel having a smaller cross-sectional area near the insertion end of the barrel and a larger cross-sectional area near the opposite end and/or a barrel having a primarily non-circular cross-sectional shape and also having one or more circular cross-sections. For example, in certain embodiments, a barrel having a generally non-circular cross-sectional shape can have greater than about 50% of the cross-sections having a non-circular cross-sectional shape, such as, for example, greater than about 60%, greater than about 70%, greater than about 80%, greater than 90%, or about 100% of the cross-section having a non-circular cross-sectional shape. In certain embodiments, substantially the entire barrel can have a generally non-circular cross-sectional shape, with the cross-sectional shape changing to circular proximal to the finger grip region or movable grip region.

The insertion end of the barrel can be open-ended or closed-ended. In certain embodiments, the insertion end of the barrel can include petals, corrugations, pleats, a film cap, or other means for covering the barrel prior to expulsion of the tampon. In certain embodiments, the feminine hygiene article can be loaded into the barrel prior to covering the insertion end of the barrel. Alternatively, the insertion end of the barrel can be covered prior to loading the feminine hygiene article into the barrel. In certain embodiments, the barrel can have an oval cross-sectional shape and the insertion end can include petals, such as, an even number of petals. In certain embodiments, the lines between petals can be parallel to the axes of the oval.

The gripping elements may be created on or in the plunger, finger grip region, or moveable grip region by any known method in the art. For example, materials such as ink, glue, polyethylene, polypropylene, polybutylene, polystyrene, polyvinylchloride, polyacrylate, polymethacrylate, polyacrylnitril, polyacrylamide, polyamide, nylon, polyimide, polyester, polycarbonate, ethylene vinyl acetate, polyurethane, silicone, derivatives thereof, copolymers thereof, or mixtures thereof, may be dispensed on the outer surface of the plunger, finger grip region, or moveable grip region by any method known in the art, such as by spraying, gluing, casting, printing, embossing, taping, or combinations thereof. Further, the gripping elements may be formed on the outer surface of the plunger, finger grip region, or moveable grip region by modifying the outer surface by any method known in the art such as, for example, molding, laser engraving, shearing, lancing, die punching, or any combinations thereof. In still other embodiments, the gripping elements may be formed by dispensing materials to and modifying the outer surface of the plunger, finger grip region, or moveable grip region.

The moveable grip region can be constructed from any suitable material. Suitable materials include, for example, polyethylene, polypropylene, polybutylene, polystyrene, polyvinylchloride, polyacrylate, polymethacrylate, polyacrylnitril, polyacrylamide, polyamide, nylon, polyimide, polyester, polycarbonate, ethylene vinyl acetate, polyurethane, silicone, derivatives thereof, copolymers thereof, mixtures thereof, paper, paperboard, cardboard, or any combinations thereof. In certain embodiments, additives can be included in the material to alter or enhance certain material properties. Suitable additives include, for example, mold release agents, slip agents, surface energy modifiers, pearlescent agents, or any other suitable additives.

The cross-section of the finger grip region or moveable grip region can be any suitable shape, such as, for example, oval, circular, arc, concave, cone convex, diamond, polygon, square, triangle, rectangular, or any combination thereof. In certain embodiments, the shape of the finger grip region, moveable grip region, or both can correspond to the barrel shape, such as an oval finger grip region or oval movable grip region with an oval barrel. Alternatively, the finger grip region, moveable grip region, or both can be a different shape than the barrel, such as, a circular finger grip region or circular movable grip region with an oval barrel.

The plunger can be constructed from any suitable material. Suitable materials include, for example, polyethylene, polypropylene, polybutylene, polystyrene, polyvinylchloride, polyacrylate, polymethacrylate, polyacrylnitril, polyacrylamide, polyamide, nylon, polyimide, polyester, polycarbonate, ethylene vinyl acetate, polyurethane, silicone, derivatives thereof, copolymers thereof, mixtures thereof, paper, paperboard, cardboard, or any combinations thereof. Examples of suitable plungers are disclosed in, U.S. Pat. No. 5,346,468 and U.S. Pat. No. 5,558,631. In certain embodiments, additives can be included in the material to alter or enhance certain material properties. Suitable additives include, for example mold release agents, slip agents, surface energy modifiers, pearlescent agents, or any other suitable additives.

The plunger can be hollow or solid. In certain embodiments, the plunger can have a hollow interior, a first end, and a second end opposed to the first end. The first end is the portion of the plunger that pushes against the tampon during the expulsion of the tampon from the barrel. The second end is the portion of the plunger in which the axial force may be applied to expel the tampon from the barrel. In certain embodiments, the plunger can have a locking mechanism, such as, a locking mechanism that retains the plunger within the barrel and/or finger grip region or movable grip region of the applicator prior to depression of the plunger and expulsion of the tampon. Examples of such locking mechanisms are described in, for example, U.S. Pat. Nos. 6,019,744 and 6,450,986.

The plunger can be any suitable shape. Suitable shapes include, for example, shapes that can work effectively to aide in the expulsion of the tampon from the tampon applicator. Suitable cross-sectional shapes include, for example circular, oval, square, rectangular, triangular, flattened circular, elliptical, and any combinations thereof. In certain embodiments, the plunger shape can correspond to the barrel shape, such as, an oval plunger with an oval barrel. Alternatively, the plunger can be a different shape than the barrel, such as, a plunger with a circular cross-section with an oval barrel. In certain embodiments, the plunger cross-sectional shape can correspond to the finger grip region or moveable grip region cross-sectional shape, such as, a circular plunger with a circular finger grip region or moveable grip region. Alternatively, the plunger can be a different cross-sectional shape than the finger grip region or moveable grip region, such as, a plunger with a circular cross-section with an oval finger grip region or moveable grip region. The plunger can also be the same cross-sectional shape as the finger grip region or moveable grip region and a different cross-sectional shape than the barrel; a different cross-sectional shape from the finger grip region or moveable grip region and the same cross-sectional shape as the barrel; or a different cross-sectional shape from the finger grip region or moveable grip region and a different cross-sectional shape from the barrel. In certain embodiments, the plunger can include more than one cross-sectional shape along the length. For example, in certain embodiments, the plunger can include a circular cross-sectional shape at one end and change to a cross-sectional shape that is the same as the cross-sectional shape of the feminine hygiene article, such as, a tampon, on the end nearest the feminine hygiene article. In certain embodiments, the plunger can maintain a cross-sectional shape along the length but the diameter of the shape can vary along the length of the plunger.

In certain embodiments, at least a portion of the applicator can contact or conform to at least a portion of the surface of the tampon. Rigid insertion end structures can be shaped in a suitable manner, such as by injection molding or by reshaping in a secondary process to provide at least a degree of profiled shape observation. Alternatively, insertion ends of applicators made from flexible or pliable materials, such as films, paper and flexible wovens or non-wovens, can also be used. Such flexible or pliable insertion ends include those which partially or fully enclose the tampon comprising a “sleeve” or a “tube,” such as, in U.S. Pat. Nos. 2,922,422 and 2,922,423; a “sheath,” such as, in U.S. Pat. Nos. 2,092,427 and 3,749,093; a “barrel,” such as, in U.S. Pat. No. 5,135,475; a “bag,” such as, in U.S. Pat. No. 3,358,686; or a “film enclosure,” such as, in U.S. Pat. No. 4,610,659.

In certain embodiments, the applicators can be designed for use with tampons that have a non-uniform expansion, such as, tampons having greater expansion in the width direction than in the length direction. Suitable tampons can have any suitable cross-sectional shape, such as, circular, generally circular, circular with flattened sides, elliptical, oval, or any other suitable cross-sectional shape. Exemplary tampons having a circular cross-section are described in, U.S. Pat. Nos. 6,837,882; 6,740,070; 6,682,513; 6,599,279; 6,554,814; and 6,258,075.

Alternatively, or in addition, the applicators can be designed for use with tampons having widthwise expansion and a non-circular cross-section, such as, a tampon having a substantially oval cross-sectional shape, such as a tampon that is formed having an oval cross-sectional shape. In certain embodiments, a generally non-circular cylindrical tampon can include a tampon having one or more circular cross-sections, wherein the tampon primarily includes non-circular cross-sectional shapes. Examples of such tampons are described in U.S. patent application Ser. No. 11/703,946, to Hasse, et al., titled “Self-Orienting Tampon Having Improved Aspect Ratio,” filed on Feb. 7, 2007.

In certain embodiments, the barrel can fully rotate with respect to the moveable grip region, such as, for example, the barrel can rotate 360° with respect to the moveable grip region. Alternatively, or in addition, the barrel can partially rotate with respect to the moveable grip region, such as, for example, the barrel can rotate less than about 360°, such as, for example, about 300°, about 240°, about 180°, about 120°, about 90°, or about 45°, or any other suitable amount. In certain embodiments, rotation of the barrel with respect to the moveable grip region can be signaled by an audible sound, such as a click.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A feminine hygiene applicator comprising:
a barrel having an outer surface and an inner surface;
a plunger having an outer surface, a withdrawal end, and an insertion end; and
wherein the outer surface of the plunger has disposed thereon or therein at least one gripping element.

2. The feminine hygiene applicator of claim 1, wherein the at least one gripping element has a form that is at least one of an embossment, tread, rib, projection, slit, groove, perforation lance, abrasive material, pressure sensitive adhesive, or high wet coefficient of friction material.

3. The feminine hygiene applicator of claim 1, wherein the at least one gripping element has a shape that is at least one of circular, oval, square, rectangular, triangular, polygonal, linear, curved, irregular, a character, a letter, symbol, or a number.

4. The feminine hygiene applicator of claim 1, wherein the at least one gripping element is raised above the outer surface of the plunger.

5. The feminine hygiene applicator of claim 1, wherein the at least one gripping element is depressed below the outer surface of the plunger.

6. The feminine hygiene applicator of claim 1, wherein the at least one gripping element comprises at least one of a logo or trademark.

7. The feminine hygiene applicator of claim 1, wherein the at least one gripping element comprises a visual signal as to where a user may place their fingers on the outer surface of the plunger.

8. The feminine hygiene applicator of claim 7, wherein the visual signal comprises the at least one gripping element having a color that differs from the color of the outer surface of the plunger.

9. The feminine hygiene applicator of claim 1, wherein the at least one gripping element comprises an indication to a user as to the distance the plunger may be withdrawn from the barrel to insert a feminine hygiene article.

10. The feminine hygiene article of claim 9, wherein the indication comprises the length of the at least one gripping element as measured from the withdrawal end of the plunger.

11. The feminine hygiene applicator of claim 1, wherein the plunger includes at least one longitudinal protrusion.

12. The feminine hygiene applicator of claim 1, wherein the outer surface of the applicator comprises a finger grip region.

13. The feminine hygiene applicator of claim 12, wherein the finger grip region comprises at least one gripping element.

14. The feminine hygiene applicator of claim 1, wherein the outer surface of the plunger, the inner surface of the barrel, or both are coated with a coating material.

15. A feminine hygiene applicator comprising:
a barrel having an outer surface and an inner surface;
a moveable grip region;
a plunger having an outer surface;
the barrel being rotatably connected to the moveable grip region;
wherein the barrel is adapted to rotate relative to the moveable grip region; and
wherein the outer surface of the plunger has disposed thereon or therein at least one gripping element.

16. The feminine hygiene applicator of claim 15, wherein the at least one gripping element has a form that is at least one of an embossment, tread, rib, projection, slit, groove, perforation lance, abrasive material, pressure sensitive adhesive, or high wet coefficient of friction material.

17. The feminine hygiene applicator of claim 15, wherein the outer surface of the plunger, the inner surface of the barrel, or both are coated with a coating material.

18. The feminine hygiene applicator of claim 15, wherein the moveable grip region comprises at least one gripping element.

19. The feminine hygiene applicator of claim 15, wherein substantially the entire barrel has a width and a thickness, with the width being greater than the thickness.

20. The feminine hygiene applicator of claim 19, wherein the barrel has a substantially oval cross-sectional shape.
